(19) United States
(12) Patent Application Publication
(10) Pub. No.: US 2022/0319716 A1
(43) Pub. Date: Oct. 6, 2022

(54) METHOD FOR EPIDEMIOLOGICAL IDENTIFICATION AND MONITORING OF A BACTERIAL OUTBREAK

(71) Applicant: BIOMERIEUX, Marcy l'Etoile (FR)

(72) Inventors: Gaël Kaneko, Marcy l'Etoile (FR); Ghislaine Guigon, Dardilly (FR)

(73) Assignee: BIOMERIEUX, Marcy l'Etoile (FR)

(21) Appl. No.: 17/626,353

(22) PCT Filed: Jul. 2, 2020
(86) PCT No.: PCT/EP2020/068611

§ 371 Date: Jan. 11, 2022

(30) Foreign Application Priority Data
Jul. 12, 2019 (EP) 19186032.9

(51) Int. Cl. G16H 50/80

(52) U.S. Cl. CPC G16H 50/80

(57) ABSTRACT

The method for detecting and monitoring a bacterial outbreak includes predicting that a collected bacterial strain and a bacterial strain from a database belong to the bacterial outbreak if their genomic distance is less than a first predetermined threshold, do not belong to the bacterial outbreak if their genomic distance is greater than a second predetermined threshold strictly greater than the first threshold, or may belong to the bacterial outbreak if their genetic distance is in-between. The first threshold is greater than or equal to a third threshold, such that a prediction that two bacterial strains with a genomic distance less than the third threshold belong to the outbreak has maximum specificity. The second threshold is less than or equal to a fourth threshold, such that a prediction that two bacterial strains with a genomic distance greater than the fourth threshold do not belong to the outbreak has maximum sensitivity.

### FIELD OF THE INVENTION

The present invention relates to the field of bacterial epidemiology, in particular the detection and monitoring of bacterial outbreaks as a function of the genomes of bacterial strains, in particular the partial or complete sequencing of the DNA and/or RNA of the bacterial strains.

### PRIOR ART

The detection of an infectious bacterial outbreak consists conventionally of determining whether several bacterial strains taken from subjects (e.g. patients and by extension animals) result from recent transmission of an identical strain among the subjects, for example transmission of the strain to several subjects from a “source” subject or transmission of the strain from subject to subject. On the basis of the classical microbiological tools, detection is usually carried out in two steps:
a. firstly suspecting a bacterial outbreak, this suspicion arising when sampled strains belong to the same bacterial species and share common phenotypic characteristics, for example an identical or similar antibiogram for the pathogenic bacteria;
b. and if suspected, conducting an epidemiological inquiry with the aim of demonstrating, or invalidating, that these strains do indeed result from transmission between subjects. This type of inquiry consists in particular of researching whether the subjects in the sampling have recently been in contact, have shared one and the same locale (e.g. one and the same operating room or one and the same room in a hospital), were cared for by one and the same caregiver, etc. This type of inquiry is generally long and painstaking, and mobilizes many people. Furthermore, an inquiry may cause considerable disturbance to the operation of an institution or a company suspected of being the object of an epidemic, since prophylactic measures are usually put in place before the end of the inquiry, for example such as putting a room or department in quarantine, or closing an operating room.

In this context, the advent of sequencing, in particular sequencing of the WGS (whole genome sequencing) type, represents a notable advance in bacterial epidemiology since a whole bacterial genome contains a level of information far greater than that delivered by the classical microbiological techniques. Not only are the criteria for deciding to launch an epidemiological study more precise, but in addition the use of genomics may also greatly simplify and normalize the latter. For example, if two strains of Staphylococcus aureus, found in samples within the same hospital department some days apart, are strictly identical from the genomic standpoint, it may be determined without additional information that the two strains do indeed form part of one and the same bacterial outbreak.

Although sequencing has proved to be a notable advance, on its own it still does not make it possible to determine the line of two bacterial strains, regardless of the species. In fact, certain bacterial species have a plastic genome that evolves very quickly in the space of a few days, and even more so if antibiotic treatment is used, so that strict identity between genomes cannot be used as the sole criterion. To take account of this plasticity, methods for detecting bacterial outbreaks consist of evaluating whether bacterial strains belong to one and the same outbreak if their genomic difference, for example calculated as a function of the number of single-nucleotide polymorphisms, is below a predetermined threshold, as described in the article “Beyond the SNP threshold: identifying outbreak clusters using inferred transmission” by J. Simson et al., December 2018, but this approach is rather imprecise owing to many sources of uncertainty, for example such as the context in which the bacteria evolve or the variability of the mutation rates as a function of the species. The authors of this article thus propose also taking into account the chronology of collection of the samples containing the bacterial strains and a priori knowledge about the mechanisms of mutation and transmission of the bacterial strains.

Besides making the epidemiological prediction models more complex, the use of a single threshold leads inevitably to a difficult compromise between sensitivity and specificity of the prediction. On the one hand, if prediction of assignment to a bacterial outbreak is too sensitive but too nonspecific, triggering of epidemiological inquiries leading to refutation of the epidemic character of an event is too frequent, which involves a considerable cost in terms of resources, operation and budget. On the other hand, if prediction of assignment to an outbreak is of low sensitivity, bacterial outbreaks are not detected, with serious consequences in terms of health, for example that of patients or consumers.

### SUMMARY OF THE INVENTION

The aim of the present invention is to propose a method for identifying and monitoring a bacterial outbreak on the basis of comparison of bacterial genomes, which offers freedom in terms of sensitivity and specificity while explicitly taking into account the sources of uncertainty in the prediction of assignment of bacterial strains to the bacterial outbreak.

For this purpose, the invention relates to a method for detecting and monitoring a bacterial outbreak linked to a bacterial species within a geographic zone, comprising:
obtaining a digital genome of a bacterial strain sampled within the geographic zone and belonging to the bacterial species;
calculating a genomic distance of the digital genome obtained with a digital genome of a database, called “epidemiological”, comprising at least one digital genome of a bacterial strain belonging to the bacterial species;
predicting:
   that the bacterial strain sampled and the bacterial strain of the database belong to the bacterial outbreak if their genomic distance is below a first predetermined threshold; or
   that the bacterial strain sampled and the bacterial strain of the database do not belong to the bacterial outbreak if their genomic distance is above a second predetermined threshold strictly higher than the first threshold; or
   that the bacterial strain sampled and the bacterial strain of the database perhaps belong to the bacterial outbreak if their genomic distance is between the first and the second threshold;
      according to said method:
the first threshold is greater than or equal to a third threshold such that a prediction that two bacterial strains having a genomic distance below the third threshold belong to the bacterial outbreak has a maximum specificity; and
the second threshold is less than or equal to a fourth threshold such that a prediction that two bacterial strains having a genomic distance above the fourth threshold do not belong to the bacterial outbreak has a maximum sensitivity.

In other words, two different thresholds are used for controlling the sensitivity and specificity of the method, the lower threshold being used for controlling the specificity of the prediction that a strain belongs to the bacterial outbreak (hereinafter “specificity of belonging”) and the higher threshold being used for controlling the sensitivity of this prediction (hereinafter “sensitivity of belonging”). The zone between these two thresholds is thus specifically provided for taking account of the uncertainties inherent in a prediction based on genomic distances. In particular, the third and fourth thresholds, applied beforehand for maximizing the specificity and sensitivity of belonging, define a zone where it is difficult to know whether strains do or do not belong to one and the same outbreak on account of data being incomplete or insufficiently diversified for learning these thresholds, ignorance of the mechanisms of mutation, which are heterogeneous within the bacterial species, imprecision of the method because of the choice of the method of genomic comparison or else errors of characterization of the foci of infection resulting from the epidemiological inquiries. This zone of uncertainty offers the user flexibility in the management of epidemics. In particular, in contrast to prediction of belonging to the bacterial outbreak, which triggers an epidemiological inquiry and prophylactic measures for curbing the bacterial outbreak, when a strain is in the intermediate zone, the user may set up a preliminary inquiry, for example crosschecking with the file of the patient from whom the sample was obtained or by analyzing their resistome, their virulome or their phylogenic position in the biodiversity of the species, for deciding whether or not a thorough epidemiological inquiry must be undertaken. Moreover, the zone between the third and fourth thresholds may in certain cases be too large, so that the prediction based on these thresholds is not optimal. The first and second thresholds, defining a zone strictly comprised between the third and fourth thresholds, allow analytical optimization of the prediction of belonging or non-belonging to the bacterial outbreak.

According to one embodiment, the first and the second thresholds are equal to two genomic distances calculated:
by constructing a learning database of digital genomes of bacterial strains belonging to the bacterial species, said database comprising:
   pairs of bacterial strains previously determined as belonging to one and the same bacterial outbreak, and tagged as “pairs of related strains”;
   pairs of bacterial strains previously determined as not belonging to one and the same bacterial outbreak, and tagged as “pairs of unrelated strains”;
by selecting a binary predictor configured for predicting that two bacterial strains are related or unrelated by comparing their genomic distance against a fifth threshold;
for each value of fifth threshold belonging to a predetermined set of values of fifth threshold, calculating
   a confusion matrix of said predictor as a function of the learning database;
   a first quality index of the predictor as a function of the confusion matrix, said first index being different than the sensitivity and specificity of the predictor;
   a second quality index, different than the first index, as a function of the confusion matrix, said second index being different than the first index, the sensitivity and the specificity of the predictor;
searching for a first value of fifth threshold that optimizes the first index and a second value of fifth threshold that optimizes the second index;
setting the first threshold equal to the minimum of the first and second values of fifth threshold and setting the second threshold equal to the maximum of the first and second values of fifth threshold.

In other words, a prediction based on a maximum specificity and specificity of belonging does not necessarily constitute an optimal prediction with respect to the available epidemiological data, stored in the learning database. By calculating the first and second thresholds that optimize the quality of the binary prediction, in fact an optimization of the management of the epidemic events is obtained, while preserving a sufficiently wide intermediate zone for continuing to alert the user of a possible bacterial outbreak.

According to one embodiment, the first index is selected for taking into account the imbalance, in the learning database, between the number of pairs of related strains and the number of pairs of related strains. In particular, the first index is the Matthews correlation coefficient or the F1 score. In general, the data concerning bacterial outbreaks, i.e. the number of strains regarded as related, are far less numerous than the strains regarded as unrelated. By using a quality index that takes this imbalance into account explicitly, better optimization of the prediction is obtained. Furthermore, the threshold corresponding to the Matthews coefficient or the F1 score favors specificity but without only taking the specificity into account.

According to one embodiment, the second index is the Youden index. This index, which takes the specificity and the sensitivity into account explicitly, allows the prediction of non-belonging to be optimized naturally, learning of which is usually carried out on an important datum. The imbalance of the database has the effect that the Youden index is more influenced by the sensitivity, the specificity being close to 1 in the entire interval between the third and fourth thresholds.

According to one embodiment, the predictor is selected in such a way that:
the true positives correspond to the pairs of related strains having a genomic distance below the fifth threshold:
the false negatives correspond to the pairs of related strains having a genomic distance above the fifth threshold;
the false positives correspond to the pairs of unrelated strains having a genomic distance below the fifth threshold; and
the true negatives correspond to the pairs of unrelated strains having a genomic distance above the fifth threshold.

According to one embodiment, the epidemiological database comprises the learning database. In other words, the learning database is supplemented as the method is applied, which makes it possible to refine the various thresholds as the database increases in size.

According to one embodiment, the genomic distance is a normalized distance. More particularly, the genomic distance between two bacterial strains is calculated by:
selecting, from a set predominantly of loci, the loci common to the digital genomes of said strains;
counting the number of allelic differences, at the common loci, between the two digital genomes of said strains;
dividing said number of differences by the number of common loci.

On normalizing by the number of loci in common, the effect of sequencing errors, in particular the fact of not identifying a locus in a bacterial strain, is attenuated.

According to a preferred embodiment, if the first and second values of fifth threshold are above 0.1, then:
the second threshold is set equal to 0.1;
the first threshold is set equal to max(D_{g}\D_{g}<0.2), where max(D_{g}\D_{g}<0.2) is the largest genomic distance, among the pairs of related strains, strictly below 0.2.

In particular, the inventors found that values above 0.1, usually obtained because a learning database is incomplete or insufficiently diverse, cause failure of learning. The inventors also noted that in the context of a suitable learning database, the first and second thresholds are less than or equal to 0.1. One of the two thresholds is thus fixed at this upper bound. In addition, the inventors found that two strains of the same subtype have, in a very great majority, a genomic distance less than 0.2. Thus, on setting the other threshold equal to max(dᵣ\dᵣ<0.2), two strains with genomic distance greater than the latter, it is predicted that these strains do not belong to the same bacterial subtype, and therefore do not belong to the same outbreak, which constitutes an important index for suspecting an epidemic. Thus, even though the data are still insufficient for accurately calculating the first and second thresholds, the user has a method at his disposal by default.

According to one embodiment, the distances between the digital genomes are calculated as a function of a database of markers, in particular a database wgMLST, cgMLST, MLST, of genes or of SNPs.

According to one embodiment, when a sampled strain is predicted as belonging to the bacterial outbreak, it is tagged in the epidemiological database as being “related” to the bacterial strains of the bacterial outbreak and as being “unrelated” to the other bacterial strains.

According to one embodiment, when a sampled strain is predicted as perhaps belonging to the bacterial outbreak, an additional characterization of said strain is carried out to determine whether it actually belongs to said outbreak, and if that is so, the sampled bacterial strain is tagged, in the epidemiological database, as being “related” to the bacterial strains of the bacterial outbreak and as being “unrelated” to the other bacterial strains.

According to one embodiment, the first and the second thresholds are recalculated regularly and/or as soon as N new strains are added to the epidemiological database, where N is an integer greater than or equal to 1.

According to one embodiment, when a strain is predicted as belonging to the bacterial outbreak, prophylactic measures are put in place to halt said outbreak.

### BRIEF DESCRIPTION OF THE FIGURES

The invention will be better understood on reading the description given hereunder, given purely as an example, and referring to the appended drawings, in which identical references denote identical elements, and in which:

FIG. 1 is a flowchart of an embodiment of the method according to the invention;

FIG. 2 shows a table of correspondence between bacterial strains stored in a learning database;

FIG. 3 is a confusion matrix of a binary predictor predicting the related or unrelated state of two bacterial strains;

FIG. 4 shows a distribution of the number of pairs of related strains and a distribution of the number of pairs of unrelated strains as a function of their genomic distance as well as a threshold Ti used for calculating the confusion matrix in FIG. 3;

FIG. 5 is a diagram illustrating different thresholds over the genomic distances used by the method according to the invention;

FIG. 6 shows a computing and sequencing system for carrying out the method according to the invention;

FIGS. 7A and 7B are distributions of the number of pairs of unrelated strains (upper distribution) and of the number of pairs of related strains (lower distribution) for the bacterial species Clostridium difficile, FIG. 7B being a magnification between 0 and 0.1 of FIG. 7A;

FIGS. 8A and 8B illustrate, for the species Clostridium difficile, the genomic distances for different optimal values of quality index, including the sensitivity, specificity, precision, accuracy (i.e. (TP+TN)/(N+P)), the F1 score, the Youden index, and the Matthews correlation coefficient, FIG. 8B being a magnification between 0 and 0.1 of FIG. 7B;

FIGS. 9A and 9B are distributions of the number of pairs of unrelated strains (upper distribution) and of the number of pairs of related strains (lower distribution) for the bacterial species Staphylococcus aureus, FIG. 9B being a magnification between 0 and 0.1 of FIG. 9A;

FIGS. 10A and 10B show, for the species Staphylococcus aureus, the genomic distances for different optimal values of quality index, including the sensitivity, specificity, precision, accuracy, F1 score, Youden index, and the Matthews correlation coefficient, FIG. 10B being a magnification between 0 and 0.1 of FIG. 10B;

### DETAILED DESCRIPTION OF THE INVENTION

Hereinafter, “below” or “less than” signifies “less than or equal to” and “above” or “greater than” signifies “greater than or equal to”, unless strictly stipulated.

An embodiment of the invention will now be described in relation to the detection and monitoring of microbiological foci of infection of a particular bacterial species in a hospital.

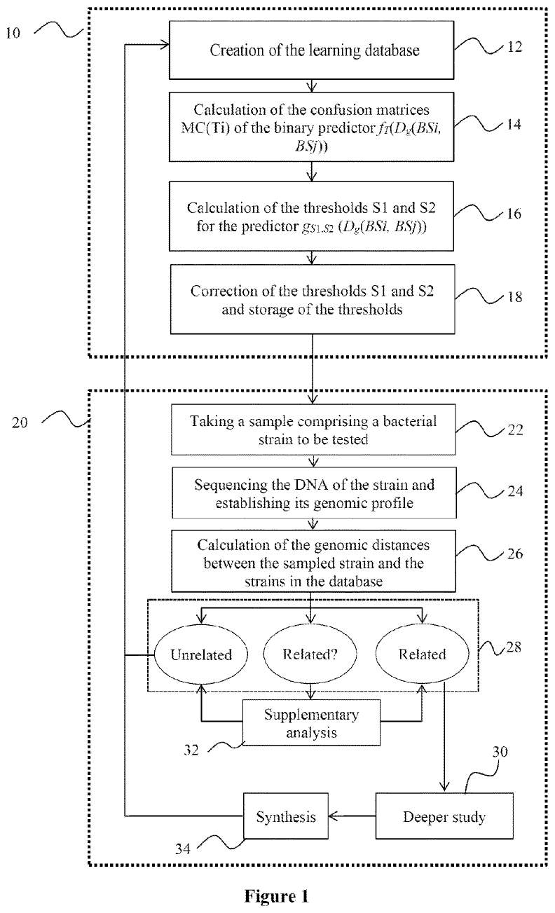

Referring to FIG. 1, this method comprises a first step 10 of learning of at least two thresholds, designated S1 and S2, on the basis of which comparisons of genomes are carried out for determining whether or not a bacterial strain belongs to a bacterial outbreak, and a second step 20 of carrying out the method according to the invention, parameterized with the thresholds learnt in step 10. More particularly, the method is based on comparison of a genomic distance, designated D_{g}(BSi,BSj), between two strains, designated BSi and BSj.

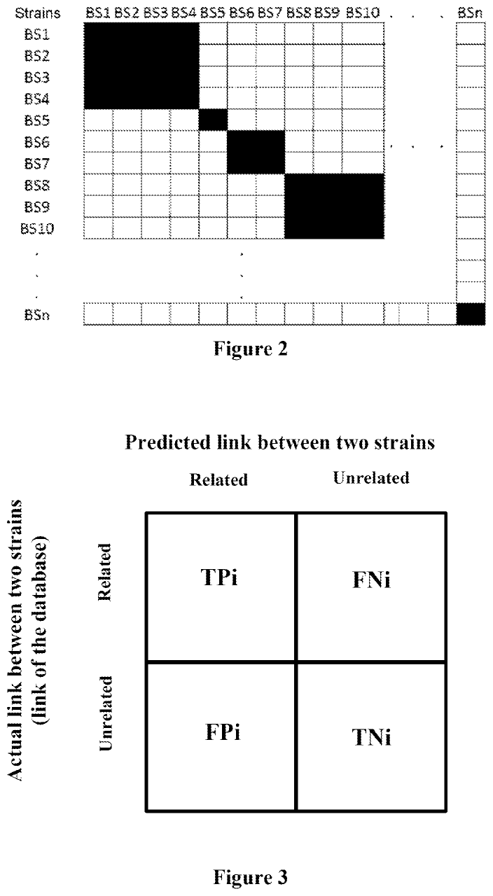

Step 10 begins with the creation, in 12, of a learning database for the species in question, comprising:
digital genomes of different strains BS1, BS2, BS3 . . . , BSN belonging to the species;
a correspondence table, illustrated in FIG. 2, linking each strain of the database to all of the other strains, where each link between two strains in the database may assume a “related” state (black boxes) when the two strains have previously been determined as belonging to one and the same bacterial outbreak, and an “unrelated” state (white boxes) when the two strains have previously been determined as not belonging to one and the same bacterial outbreak, the state of the link between two strains being determined for example during a prior epidemiological study. Furthermore, the link of a strain relative to itself is fixed in the “related” state. As can be seen in FIG. 2, several foci of infection for the species in question may be taken into account for determining the “related” and “unrelated” states of the strains in the learning database. As will be described hereunder, the learning database may also contain strains determined as being “related” but without being diagnosed as belonging to any bacterial outbreak.

Preferably, said table also stores the genomic distances D_{g}(BSi,BSj) between each pair of strains BSi and BSj of the learning database;
a table that lists all of the foci of infection identified with their associated strains;
the resistomes (set of genetic markers contributing to a bacterium's sensitivity or resistance to antibiotics) and the virulomes (set of genetic markers contributing to the virulence of a bacterium) of the strains BS1, BS2, BS3 . . . , BSN.

The genome of a bacterial strain is preferably obtained by:
taking a sample from a patient comprising the strain;
preparing an isolate of the strain, for example by spreading the sample on an agar culture medium and incubating to grow a colony of the bacterial strain;
taking a part of the colony and preparing the quantity taken for sequencing (e.g. lysis to release the DNA of the bacteria, if necessary amplification of the DNA released and preparing a library for the sequencing techniques requiring it);
sequencing, preferably complete (or WGS sequencing), of the DNA so as to produce digital sequences, commonly called “reads”, for example using technology of the “next generation sequencing” type, such as with the “MiSeq” sequencing platform from the company Illumina Inc., San Diego, Calif.;
optionally, assembly of the reads so as to produce assembled sequences, known by the term “contig”;
characterization, by the wgMLST (“whole genome multilocus sequencing typing”) technique, of the genome in the form of contig or of reads, commonly called “wgMLST profile”. As is known per se, this characterization consists of locating the loci in the genome from a predetermined set of loci, and for each locus identified, determining the allele that represents this locus. The wgMLST technique is described for example in the document “MLST revisited: the gene-by-gene approach to bacterial genomics” by Martin C. J. Maiden, Nature Reviews Microbiology, 2013.

Learning continues by calculating thresholds S1 and S2 as a function of the learning database. More particularly, this calculation consists of transforming:
a first predictor f_{T} of belonging or non-belonging of two strains to a bacterial outbreak based on a single threshold T over the genomic distances D_{g}(BSi,BSj) dividing the space of the genomic distances into just two intervals:

$${f}_{T} ( {D}_{g} ( BSi , BSj ) ) = \{ \begin{matrix}1 & if the strains BSi and BSj are related \\ - 1 & if the strains BSi and BSj are unrelated\end{matrix}$$

into a second predictor g_{S1,S2} of belonging or non-belonging of two strains to a bacterial outbreak based on two thresholds S1 and S2 over the genomic distances D_{g}(BSi,BSj) dividing the space of the genomic distances into three intervals:

$${g}_{S 1 , S 2} ( {D}_{g} ( BSi , BSj ) ) = \{ \begin{matrix}1 & if the strains BSi and BSj are related \\ 0 & if the strains BSi and BSj are potentially related \\ - 1 & if the strains BSi and BSj are unrelated\end{matrix}$$

In a preferred variant, the first predictor f_{T} is defined such that:

$$\{ \begin{matrix}if {D}_{g} ( B Si , BSj ) \leq T & then {f}_{T} = 1 \\ if {D}_{g} ( B Si , BSj ) > T & then {f}_{T} = - 1\end{matrix}$$

and the second predictor is defined such that:

$$\{ \begin{matrix}if {D}_{g} ( B Si , BSj ) \leq S 1 & then {g}_{S 1 , S 2} = 1 \\ if S 1 < {D}_{g} ( B Si , BSj ) \leq S 2 & then {g}_{S 1 , S 2} = 0 \\ if {D}_{g} ( B Si , BSj ) > S 2 & then {g}_{S 1 , S 2} = - 1\end{matrix} \begin{matrix} \\ \\ \end{matrix}$$

Preferably, the genomic distance D_{g}(BSi,BSj) is a normalized distance, and therefore between 0 and 1, calculated by:
a. identifying, in the wgMLST profiles of the two strains BSi and BSj, the loci that they have in common;
b. for each common locus, determining whether there is an allelic difference between the two strains, and in this case incrementing by 1 a counter Compt of allelic differences if at least one allelic difference is found;
c. calculating D_{g}(BSi,BSj) from the following formula, where N_{lc} is the number of loci in common:

$${D}_{g} ( B S i , B S j ) = \frac{Compt}{{N}_{lc}}$$

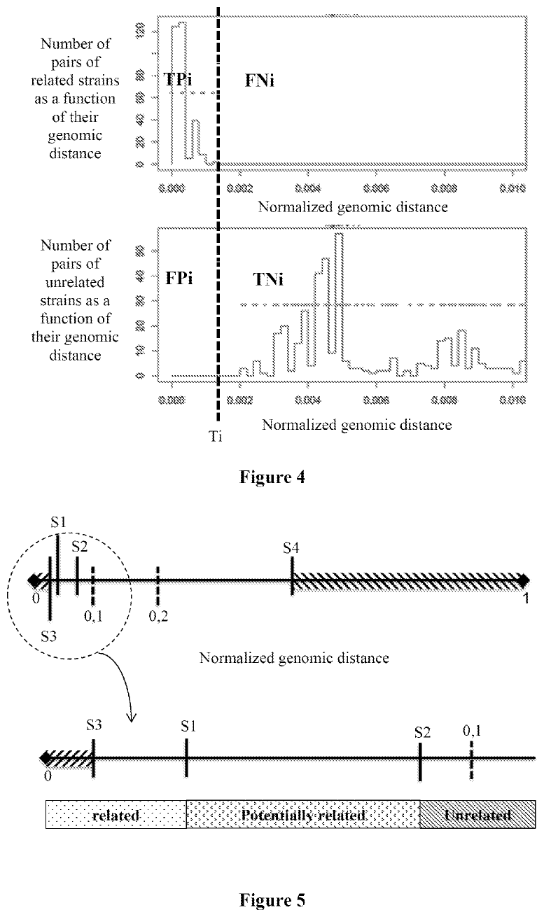

Calculation of the thresholds S1 and S2 begins, at 14, by calculating a confusion matrix MC(Ti) of the binary predictor f_{T} for each of the values Ti of a set {T1, T2, . . . , TM} of values of thresholds T between 0 and 1, for example with an increment of 10⁻⁴. Calculation of the confusion matrix (Ti), illustrated in FIG. 3, for the threshold Ti is shown in FIG. 4 and consists of counting:
the true positives, designated “TPis”, equal to the total number of pairs of related strains in the database such that D_{g}(BSi,BSj)≤Ti;
the false negatives, designated “FNi”, equal to the total number of pairs of related strains in the database such that D_{g}(BSi,BSj)>Ti;
the false positives, designated “FPi”, equal to the total number of pairs of unrelated strains in the database such that D_{g}(BSi,BSj)≤Ti; and
the true negatives, designated “TNi”, equal to the total number of pairs of unrelated strains in the database such that D_{g}(BSi,BSj)≤Ti.

Once the set of confusion matrices {MC(T1), MC (T2, . . . , MC (TM))} has been calculated, the method continues, at 16, with calculation of different thresholds illustrated in FIG. 5:
a threshold S3 such that the specificity of the predictor f_{T} is maximum, and therefore such that the specificity of the prediction that two strains are related is maximum, i.e.

$$S 3 = arg {max}_{Ti} ( \frac{TNi}{N} ) ,$$

where N is the number of pairs of unrelated strains;
a threshold S4 such that the sensitivity of the predictor f_{T} is maximum, and therefore such that the sensitivity of the prediction that two strains are unrelated is maximum, i.e.

$$S 4 = arg {max}_{Ti} ( \frac{TPi}{P} ) ,$$

where P is the number of pairs of related strains;
the threshold S1 optimizing a first quality index of the predictor f_{T}, different than the sensitivity and the specificity and explicitly taking into account the imbalance between the numbers P and N, preferably the Matthews correlation coefficient (MCC), i.e.

$$S 1 = arg {max}_{Ti} ( \frac{TPi \cdot TNi - FPi \cdot FNi}{\sqrt{( TPi + FPi ) \cdot ( TPi + FNi ) \cdot ( TNi + FPi ) \cdot ( TNi + FNi )}} ) ;$$

the threshold S2 optimizing a second quality index of the predictor f_{T}, different than the sensitivity and the specificity, preferably the Youden index, i.e.

$$S 2 = arg {max}_{Ti} ( \frac{TPi}{P} + \frac{TNi}{N} - 1 ) .$$

A step 18 of inspecting the quality of the thresholds S1 and S2 is then carried out. More particularly (the sign “\” signifying “such that”):
if the thresholds S1 and S2 are less than or equal to 0.1, they are saved, signifying that the learning database is suitable for their calculation and their subsequent use;
if the thresholds S1 and S2 are above 0.1 or differ by less than 1%, then their values are fixed at 0.1 and M=max(D_{g}(BSi,BSj)\(D_{g}(BSi,BSj)<0.2), where max(D_{g}(BSi,BSj)\(D_{g}(BSi,BSj)<0.2) is in this case the maximum genomic distance that is closest to 0.2 among the pairs of related strains in the learning database;
if one of the thresholds S1 or S2 is greater than 0.1, this threshold is then fixed at the minimum of the value 0.1 and max(D_{g}(BSi,BSj)\(D_{g}(BSi,BSj)<0.2) if this minimum value is different than the other threshold (e.g. differs by more than 1%), otherwise this threshold is fixed at the maximum of these two values.

For the sake of simplification, it will be assumed hereinafter that the threshold S1 is below the threshold S2, so that, as illustrated in FIG. 4, these thresholds divide the space of the genomic distances into three intervals:
a lower interval]0, S1]. If the genomic distance between two strains is comprised within this interval, these strains are predicted as being “related” (g_{S1,S2}=1);
an upper interval]S2,1]. If the genomic distance between two strains is comprised within this interval, these strains are predicted as being “unrelated” (g_{S1,S2}=−1); and
an intermediate interval]S1, S2]. If the genomic distance between two strains is comprised within this interval, these strains are predicted as “potentially related” (g_{S1,S2}=0).

The thresholds S1 and S2 are then stored in a computer memory of a computer system used for carrying out step 20 now described, said system further comprising the learning database. Step 20, which takes place within the hospital for detecting and monitoring epidemics of a bacterial nature, is for example carried out systematically as soon as a patient is affected by a bacterial infection, an environmental sample comprises a pathogenic bacterium or a patient presents with symptoms identical or similar to another patient within the hospital. Other criteria may of course be used for starting this step.

Step 20 begins, at 22, with the taking of a sample containing the pathogenic strain, if this sampling has not yet taken place, then continues, at 24, with sequencing of the strain and establishing its wgMLST profile as described in connection with step 12. At 26, the genomic distance D_{g}(BSi,BSj) between the sampled strain and each of the strains in the learning database is then calculated. A first epidemiological diagnosis is then issued at 28. More particularly:
if the sampled strain is not related to any strain in the database, i.e. whatever the strain in the database, the genomic distance D_{g}(BSi,BSj) from the sampled strain is above the threshold S2, then it is determined that the sampled strain does not belong to any bacterial outbreak;
if the sampled strain is related to a strain in the database, i.e. these two strains have a genomic distance D_{g}(BSi,BSj) less than or equal to the threshold S1, an alarm is triggered for the user's attention and a deeper epidemiological study 30 is started, as well as, if applicable, prophylactic measures for combating transmission of the strain sampled within the hospital;
if the sampled strain is potentially related to a strain in the database (designated “related?” in FIG. 1), i.e. if these two strains have a genomic distance D_{g}(BSi,BSj) between the thresholds S1 and S2, a supplementary analysis is carried out, at 34, for removing the uncertainty about the link between these two strains. Preferably, the resistome and the virulome of the sampled strain are determined and then compared with the resistome and the virulome of the strain to which it is potentially related. If the resistomes and the virulomes are in agreement, the strains are then determined as being related, the alarm is triggered and the deeper study 30 is carried out. Otherwise the strains are determined as being unrelated. Finally, in the case when this comparison does not settle the matter, the deeper study 30 is carried out. Other data may be used in this supplementary study, for example such as the time elapsed between taking of the sample and that of the strain in the database, the number of different SNPs in the plastic genes, etc.

As is known per se, one of the objectives of study 30, conducted by the hospital's epidemiology team, is to determine whether different strains sampled within the hospital constitute an epidemic. At the end of this study, the link between different strains is established definitively, namely “related” or “unrelated”. Moreover, if an epidemic is detected, then the strains of the epidemic are also tagged as a function of this epidemic. The genome, the wgMLST profiles, the resistome and the virulome of the sampled strain, its links with the other strains in the database as well as the data concerning the bacterial outbreak are then stored in the learning database so as to be able to be used subsequently. The thresholds S1 and S2 may thus be updated regularly or at each new entry in the database in order to refine their values.

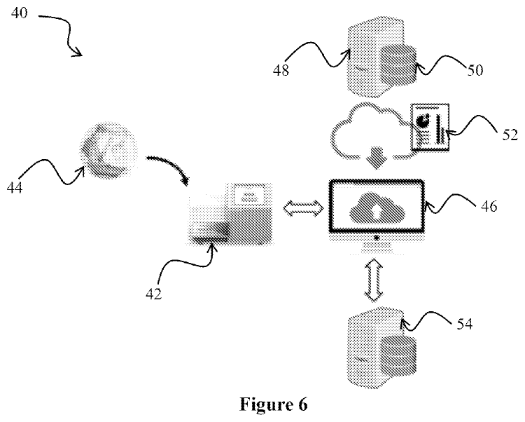

FIG. 6 illustrates a computing and sequencing system 40 for carrying out the method according to the invention. The system 40 comprises a sequencing platform 42 for sequencing the bacterial DNA of a sample 44 and thus producing a set of digital sequences, or “reads”. The platform 42 is connected to a data processing unit 46, for example a personal computer, which receives the sequences, and optionally applies a program for assembly of the reads to produce the contigs. Moreover, unit 46 is connected to a remote server 48 using software as a service (or “Saas”), for example in the form of a cloud solution. Unit 46, on which “front end” software runs, sends to the server 48 the genomes sequenced by the platform 42 in the form of reads or contigs. The server 48, on which the information service runs in the form of “back end” and which is connected to the learning database 50, receives the genomes and carries out the processing steps of the method according to the invention (e.g. steps 14-18 and 24-32 in FIG. 1), the server storing in a computer memory the set of instructions necessary for carrying this out. The server returns the results of the processing to unit 46 in the form of a report 52. The system 40 also comprises one or more servers 54 connected to unit 42, these servers being in particular those of the computer system storing the patient and epidemiological data, these data being used in the deeper studies for characterizing the epidemiological bacterial outbreaks.

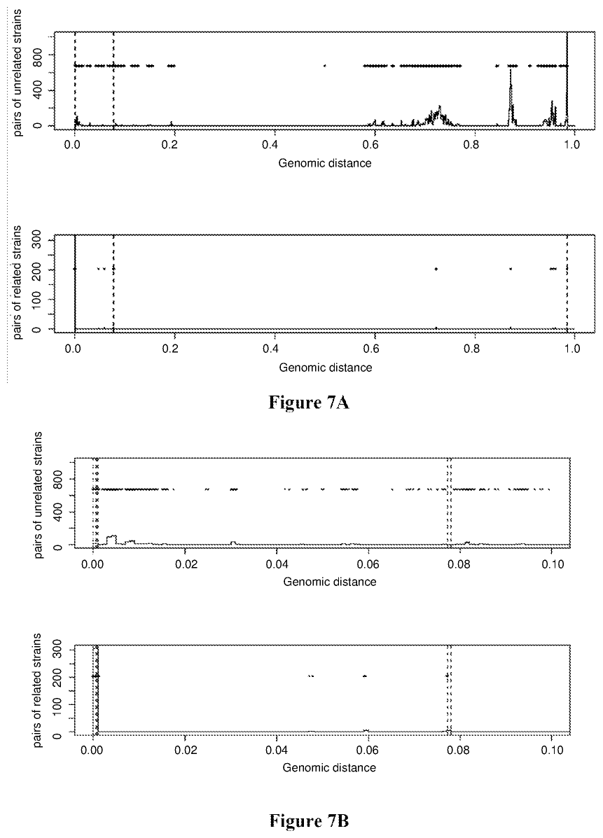

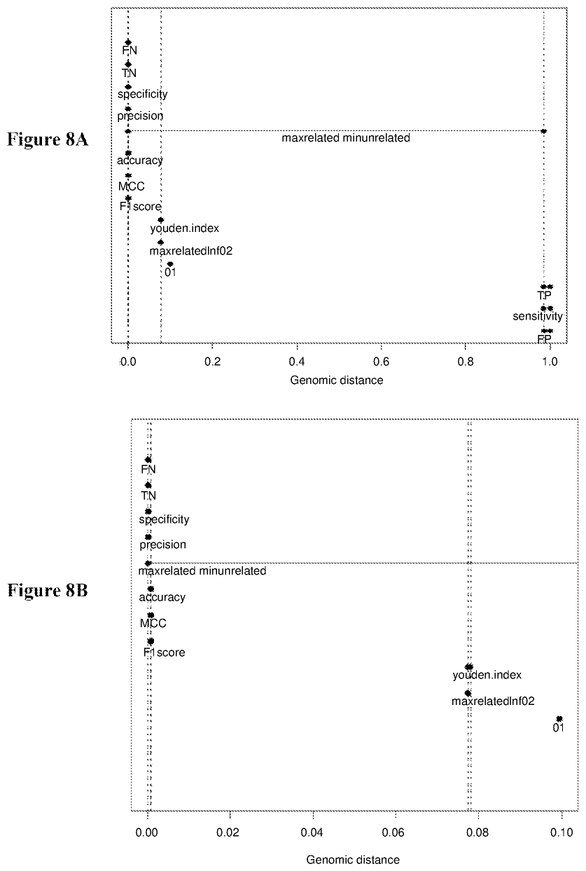

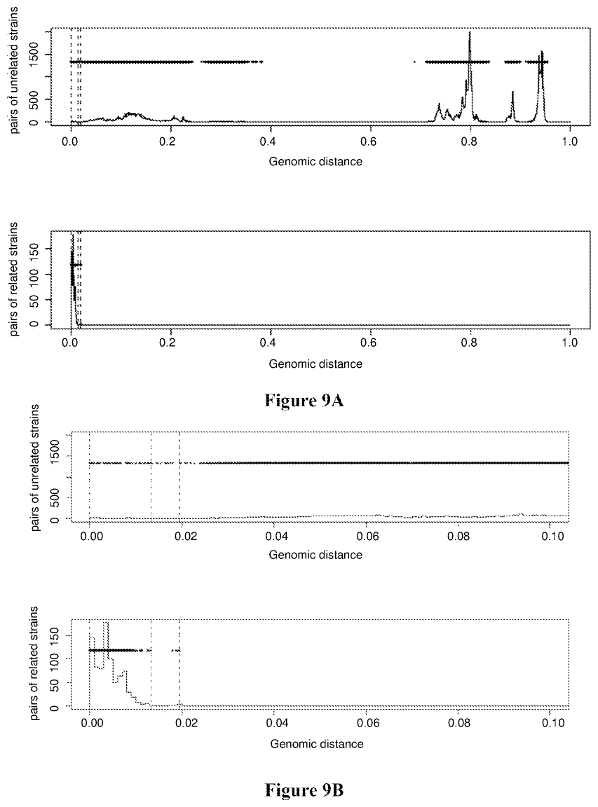

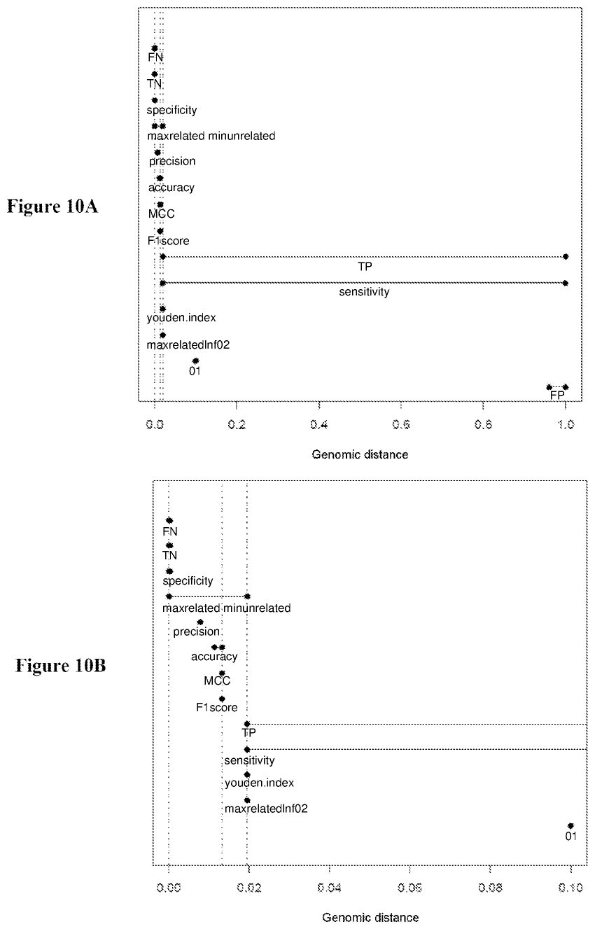

FIGS. 7 and 9 illustrate distributions of the number of pairs of related strains and of unrelated strains respectively for the species Clostridium difficile (FIGS. 7A and 7B) and Staphylococcus aureus (FIGS. 9A and 9B). As can be seen from these figures, there are pairs of related strains whose genomic distance is large (for example beyond 0.6 for Clostridium difficile) and pairs of unrelated strains whose genomic distance is small (for example below 0.2 for Staphylococcus aureus). Thus, a zone exists in which a genomic distance could code both for the “related” state or the “unrelated” state if a single threshold was used. This intermediate zone is present naturally and corresponds for example to strains belonging to one and the same subtype but that have not been judged as belonging to one and the same bacterial outbreak. Moreover, it is observed from FIGS. 8A-B and 10A-B that on selecting the thresholds S3 (maximum specificity, designated “specificity”) and S4 (maximum sensitivity, designated “sensitivity”) for dividing the space of the genomic distances into three, the intermediate zone is so large that a good number of strains would be judged as potentially related. Using the thresholds S1 (e.g. maximizing the Matthews coefficient MMC) and S2 (e.g. optimizing the Youden index), which optimize the quality of the prediction, it is noted that the intermediate zone is reduced appreciably while maintaining very good overall sensitivity.

An application to the epidemiology of pathogenic bacteria within a hospital has been described. Of course, the invention is not limited to this application and may be used in the field of industrial (for example control in the food industry), environmental, and veterinary microbiological control.

The use of wgMLST profiles for calculating the genomic distances has been described. Other profiles may be used, for example such as cgMLST (“core genome multilocus sequencing typing”) profiles, MLST, sets of SNPs or of genes.

The use of the Youden index and of the Matthews correlation coefficient has been described. Other quality indices may be used, for example such as the F1 score (i.e. 2 TP/(2TP+FP+FN)), the coefficient χ₁, the accuracy (i.e. (TP+TN)/(N+P)), precision (i.e. TP/(TP+FP)). Preferably, at least 1 of these indices takes account of the imbalance of the database.

A learning database, also used for comparing with sampled strains, has been described. As a variant, a separate database, or “epidemiological database”, may be used for processing the sampled strains. Such a database is for example suitable for a hospital, an institution, a company etc., and the learning database is then only used for establishing the values of the thresholds.

1. A method for detecting and monitoring a bacterial outbreak linked to a bacterial species within a geographic zone, comprising:
obtaining a digital genome of a bacterial strain sampled within the geographic zone and belonging to the bacterial species;
calculating a genomic distance of the digital genome obtained from a digital genome of a database, called epidemiological database, comprising at least one digital genome of a bacterial strain belonging to the bacterial species;
predicting:
(i) that the bacterial strain sampled and the bacterial strain of the epidemiological database belong to the bacterial outbreak if their genomic distance is below a first predetermined threshold; or
(ii) that the bacterial strain sampled and the bacterial strain of the epidemiological database do not belong to the bacterial outbreak if their genomic distance is above a second predetermined threshold strictly higher than the first threshold; or
(iii) that the bacterial strain sampled and the bacterial strain of the epidemiological database possibly belong to the bacterial outbreak if their genomic distance is between the first and the second threshold;
wherein
the first threshold is greater than or equal to a third threshold so that, if two bacterial strains have a genomic distance below the third threshold, the prediction, (i) that the two bacterial strains belong to the bacterial outbreak has a maximum specificity; and
the second threshold is less than or equal to a fourth threshold so that, if two bacterial strains have a genomic distance above the fourth threshold, the prediction (ii) that the two bacterial strains do not belong to the bacterial outbreak has a maximum sensitivity.

2. The method as claimed in claim 1, wherein the first and the second thresholds are equal to two genomic distances calculated by:
constructing a learning database of digital genomes of bacterial strains belonging to the bacterial species, the learning database comprising:
(i) pairs of bacterial strains previously determined as belonging to one and the same bacterial outbreak, and tagged as pairs of related strains;
(ii) pairs of bacterial strains previously determined as not belonging to one and the same bacterial outbreak, and tagged as pairs of unrelated strains;
selecting a binary predictor configured for predicting that two bacterial strains are related or unrelated by comparing their genomic distance against a fifth threshold;
for each value of fifth threshold belonging to a predetermined set of values of fifth threshold, calculating
(i) a confusion matrix of the binary predictor as a function of the learning database;
(ii) a first quality index of the binary predictor as a function of the confusion matrix, the first quality index being different than a sensitivity and specificity of the binary predictor;
(iii) a second quality index, different from the first quality index, as a function of the confusion matrix, the second quality index being different from the first quality index, of the sensitivity and specificity of the binary predictor;
finding a first value of fifth threshold that optimizes the first quality index and a second value of fifth threshold that optimizes the second quality index;
setting the first threshold equal to a minimum of the first and second values of fifth threshold and setting the second threshold equal to a maximum of the first and second values of fifth threshold.

3. The method as claimed in claim 2, wherein the first index is selected for taking into account an imbalance, in the learning database, between a number of the pairs of related strains and a number of the pairs of related strains.

4. The method as claimed in claim 3, wherein the first quality index is a Matthews correlation coefficient or a F1 score.

5. The method as claimed in claim 2, wherein the second quality index is a Youden index.

6. The method as claimed in claim 2, wherein the binary predictor is selected so that:
true positives correspond to pairs of related strains having a genomic distance below the fifth threshold:
false negatives correspond to pairs of related strains having a genomic distance above the fifth threshold;
false positives correspond to pairs of unrelated strains having a genomic distance below the fifth threshold; and
true negatives correspond to pairs of unrelated strains having a genomic distance above the fifth threshold.

7. The method as claimed in claim 2, wherein the epidemiological database comprises the learning database.

8. The method as claimed in claim 2, wherein the genomic distance is a normalized distance.

9. The method as claimed in claim 8, wherein the genomic distance between two bacterial strains is calculated by:
selecting, in a set predominantly of loci, a loci common to the digital genomes of the strains;
counting a number of allelic differences, at the common loci, between the two digital genomes of the strains;
dividing the number of differences by the number of common loci.

10. The method as claimed in claim 9
wherein the first quality index is a Matthews correlation coefficient or a F1 score, the second quality index is a Youden index, or both the first quality index is a Matthews correlation coefficient or a F1 score and the second quality index is a Youden index,
and wherein, if the first and second values of fifth threshold are above 0.1, then:
the second threshold is set equal to 0.1;
the first threshold is set equal to max(D_{g}\D_{g}<0.2), where max(D_{g}\D_{g}<0.2) is a largest genomic distance, among the pairs of related strains, strictly below 0.2.

11. The method as claimed in claim 1, wherein the distances between the digital genomes are calculated as a function of a database of markers.

12. The method as claimed in claim 1, wherein, when a sampled strain is predicted as belonging to the bacterial outbreak, the sampled strain is tagged in the epidemiological database as being related to the bacterial strains of the bacterial outbreak and as being unrelated to the other bacterial strains.

13. The method as claimed in claim 1, wherein, when a sampled strain is predicted as perhaps belonging to the bacterial outbreak, an additional characterization of the sampled strain is carried out to determine whether the sampled strain actually belongs to the bacterial outbreak, and if that is so, the sampled bacterial strain is tagged, in the epidemiological database, as being related to the bacterial strains of the bacterial outbreak and as being unrelated to the other bacterial strains.

14. The method as claimed in claim 1, wherein the first and the second threshold are recalculated regularly.

15. The method as claimed in claim 1, wherein, when a strain is predicted as belonging to the bacterial outbreak, prophylactic measures are put in place to halt the bacterial outbreak.

16. The method as claimed in claim 11, wherein the database of markers is a database wgMLST, cgMLST, or MLST.

17. The method as claimed in claim 1, wherein the distances between the digital genomes are calculated as a function of a database of genes.

18. The method as claimed in claim 1, wherein the distances between the digital genomes are calculated as a function of a database of SNPs.

19. The method as claimed in claim 11, wherein the first and the second threshold are recalculated as soon as N new strains are added to the epidemiological database, where N is an integer greater than or equal to 1.

20. The method as claimed in claim 1, wherein the first and the second threshold are recalculated as soon as N new strains are added to the epidemiological database, where N is an integer greater than or equal to 1.
